(19) United States
(12) Patent Application Publication
(10) Pub. No.: US 2007/0016104 A1
(43) Pub. Date: Jan. 18, 2007

(54) Analysis appliance for analysis of blood samples

(76) Inventors: Paul Jansen, Mannheim (DE); Jurgen Rasch-Menges, Schwetzingen (DE); Wilfried Schmid, Mannheim (DE)

Correspondence Address: BOSE MCKINNEY & EVANS LLP, 2700 FIRST INDIANA PLAZA, 135 NORTH PENNSYLVANIA STREET, INDIANAPOLIS, IN, 46204, US

(21) Appl. No.: 11/490,393

(22) Filed: Jul. 20, 2006

Related U.S. Application Data
(63) Continuation of application No. PCT/EP05/00394, filed on Jan. 17, 2005.

(30) Foreign Application Priority Data
Jan. 20, 2004 (DE) 10 2004 002 874.5

(51) Int. Cl. A61B 5/00

(52) U.S. Cl. 600/583; 600/584

(57) ABSTRACT

The invention relates to an analysis appliance for analysis of blood samples, and to a user identification method integrated in the analysis appliance. The analysis appliance comprises a sampling device for taking blood samples, and an analysis device. A delivery device is also provided for transferring the blood samples from the sampling device to the analysis device, wherein the operating parameters concerning sampling, transfer of the sample to the analysis device, and analysis of the sample, can be adapted to the respective user. The operating parameters can be adapted by automatic, spontaneous execution of an automatically determined number of test measurements.

### RELATED APPLICATIONS

This is a continuation application of International Application PCT/EP2005/000394, which claims priority to DE 10 2004 002 874.5, filed Jan. 20, 2004.

### BACKGROUND

The invention relates to an analysis appliance for analysis of blood samples, and more particularly, an analysis appliance of the type comprising a sampling device for taking blood samples, an analysis device, and a delivery device for transferring the blood samples from the sampling device to the analysis device, in which the operating parameters concerning sampling, transfer of the samples to the analysis device, and analysis of the samples, can be adapted to the user.

Glucose meters of the type in question are known in many configurations. The analysis appliance of the generic type is a non-integrated appliance in which the required volume of blood sample is applied manually by the user. The drop of blood is applied to a test field or to a capillary. To assist this process, various puncturing aids with exchangeable lancets are known.

Glucose meters are known in which the blood is collected automatically. In these known appliances, a lancet is introduced into the skin and the volume of blood is collected in most cases under vacuum. This method is crude and requires a deep puncture, which causes considerable pain to the user. Additionally, the user must handle these devices correctly in order to permit proper use of the appliance. The success of the blood collection is thus not always guaranteed due to user error. A further factor is that these known appliances often require a painful use of the lancet in order to puncture the skin.

WO 02/100251 A2 discloses an appliance with a lancet which can be introduced into the user's skin under software control. To operate this appliance, a user can choose from a large number of parameters which are provided from the outset and which in the end control the movement of the lancet into the skin. In addition, this known appliance allows the user to enter data which are specific to the user and are processed by appropriate software. A problem with this appliance is that the user in some circumstances inputs parameters which lead to particularly painful insertion of the lancet or to too great a flow of blood. The known appliance allows the possibility of adapting already stored parameters to the parameters input by the user, but this still involves the risk of the user inputting incorrect parameters when the appliance is used for the first time, or of parameters unsuitable for the user being read from a database. The successful operation of this appliance is to this extent uncertain, especially when it is being used for the first time, and it requires special knowledge on the part of the user concerning how to enter the parameters specific to him.

To this extent, in the known appliances, the depth of insertion of the lancet can be adapted to the needs and to the anatomy of the user only at the cost of considerable problems. Moreover, parameters such as the speed of expulsion of blood, the contact pressure and the duration of pressure cannot be optimally determined, and they require experience which can come only from using the known glucose meter several times. In this connection, there is always the danger that, upon renewed use of the glucose meter, the user will set a parameter incorrectly.

### SUMMARY OF THE INVENTION

The present invention provides an analysis appliance of the type mentioned at the outset that can be optimally adapted to the physical conditions set by each user who uses the device.

In one form thereof, the present invention provides an analysis appliance for analysis of blood samples comprising a sampling device for taking blood samples, an analysis device, a delivery device for transferring the blood samples from the sampling device to the analysis device, and at least one pressure sensor for sensing and recording a pressure profile of a particular user. The analysis appliance is configured to compare the recorded pressure profile with a predetermined pressure profile stored in a database for the particular user.

In an exemplary form thereof, the analysis appliance according to the invention is configured such that the operating parameters can be adapted by automatic, spontaneous execution of an automatically determined number of test measurements. The analysis appliances according to exemplary embodiments of the invention are preferably portable appliances for glucose determination, for example, portable glucose meters.

According to one exemplary embodiment, it has been found that the measurement device which performs a large number of test measurements in a very short time is able to determine the physical conditions prevailing at the time of each use. In addition, it has been found that, even when it is being used for the first use, the user is not directed to set defined parameters, and instead the analysis appliance, by performing the test measurements, automatically learns which operating parameters are to be set. To this extent, with an analysis appliance according to this exemplary embodiment of the invention, it is possible, from a large number of operating parameters, to always find the correct ones adapted to the user. In a next step, it has been found that an automatically determined number of test measurements permits an approximation of the values of the operating parameters to the optimal value. To this extent, the user is free in the use of the analysis appliance and is able to use the latter without entering technical parameters. Incorrect settings are largely ruled out.

The results of test measurements can be determined by software control and can be stored in a database and then retrieved again. It is also conceivable for the software, based on the generated parameters from the test measurements, to recognize the part of the body preferably used by the user (e.g. finger pad, ball of thumb, forearm, etc.) and the user-specific application pressure and the angle at which the appliance is held.

In addition, it is conceivable to determine the length of time to expulsion of blood and also the volume of the expelled blood and to make use of this information in future operating parameters. Through this specific embodiment, it is possible for the analysis appliance to be used by different users, in which case a set of data specific to each user can be stored and can then be accessed upon renewed measurements.

The start of the test measurements can be triggered manually. It is conceivable for user messages or instructions to appear, for example, on a display in the analysis appliance in order to show that the appliance is ready to use. Instead of this, it is also possible for the trigger to be a pressure-sensitive actuating member, for example. This specific exemplary embodiment is advantageous because the user must inevitably press a glucose meter, for example, against his body in order to obtain blood. The actuating member can be designed as a cone or in another configuration adapted to the conditions in the area of the body where the glucose meter is applied. The actuating member is connected to one or more mechanical switches which close a contact when pressure is applied to the actuating member. The actuating member can rest on an elastic abutment which can preferably be designed as a spring, for example a helical spring, a leaf spring or a pneumatic spring. Springs represent extraordinarily reliable mechanical aids whose spring constant can serve as a measure of the pressure applied. To this extent, with suitable choice of spring constant, it is possible to rule out the possibility of test measurements taking place when the pressure is too low.

Pressure sensors for recording a pressure profile can be assigned to the actuating member or to the switches. Recording a pressure profile permits identification of a user through the characteristic pressure profile specific to him. If several pressure sensors are used, then these can also identify the area of the body and its curvature and, consequently, the user, if the different curvature of a fixed part of the body, for example on the arm or forearm, finger, or ball of the thumb, an individual pressure and optional position detection.

The sampling device can be optionally activated by pressure applied to the actuating member. This ensures that the blood sample is taken only after a defined pressure is applied. This avoids inadvertent injuries which occur when a sampling device is activated undesirably.

The analysis appliance can have a measurement device for comparing a predetermined pressure profile with an applied pressure. This ensures that a characteristic pressure profile can be allocated to a very specific user, to whom, for example, the glucose meter can be adapted also in respect of the other operating parameters.

The sampling device can be controlled by the measurement device. This ensures that a sample is taken only when a characteristic pressure profile is detected. Consequently, it is ensured that the sampling procedure is adapted exactly to the respective user.

The sampling device can comprise a lancet for puncturing the skin. This embodiment is commercially advantageous since lancets are readily available and have for years been a customary and reliable means of puncturing skin.

The sampling device comprises means for detecting the flow of blood. These can, for example, be designed as a capillary arrangement for through-flow measurement, with suitable sensors, for example optical sensors, as a chemical test field, for example, to be evaluated by reflectometry, as a sensor to be evaluated electrochemically, or as a test field to be evaluated by means of a CCD camera. In this connection, it is conceivable for the sampling parameters, in particular the depth of insertion of the lancet, to be regulated by data on the speed of blood expulsion detected by said means. This specific embodiment ensures that the sampling procedure, in particular the puncturing by the lancet, is optimized as a function of the blood flow, thus largely avoiding too deep an insertion and associated pain. The optical sensors can comprise a measurement device which reacts to a chemical color reaction on a test field. From the intensity of coloring of the test field, or from the detection of a color change, it is then possible to compute the volume of blood applied. To this extent, a particularly effective and defined sampling procedure is permitted. The blood passes into a capillary, a sensor being assigned to the capillary and detecting whether blood has been taken up. A second sensor could be assigned to a test field on which a further sensor checks whether blood has flowed through the whole of the test area.

The expelled volume of blood can also be recorded by a camera which conveys the recorded image and detected data concerning the speed of expulsion of the blood to software which evaluates the image and determines the volume of blood expelled in a unit of time.

It is thus possible to ascertain whether blood is flowing and, if appropriate, the extent of the volume of blood expelled can be determined over time.

According to a further aspect of the present invention, the adaptation of the operating parameters to different users by identification of the respective user can be done selectively and automatically. In this way, it is advantageously ensured that different users cannot be punctured with a single lancet. In this way, risks of infection can be ruled out. In addition, a glucose meter is in this way not limited in its use to an individual user, and instead can be made available to different users. A very particular advantage here is that the analysis appliance can differ between individual users and can enforce a change of lancet.

The software can be controlled by user interfaces. A safety feature in this respect is that the software can be activated by user identification. The precautionary measure of providing a user interface for performing user identification is advantageous in that unauthorized persons are excluded from using a glucose meter, for example. This effectively prevents pathogens from being introduced into a circle of users. After user identification has been completed, if appropriate by a password, a set of data specific to the user is loaded. It is also conceivable that dialogues with the user are made possible via the user interfaces. It is additionally conceivable for the user to be able to enter user-specific data in order to modify sets of data specific to him. Finally, it is conceivable for the user interface to output measurement values, data, time and flags.

The operating parameters are adapted by software which communicates with suitable hardware. The software accesses databases in which the operating parameters of previous measurements are stored. In this way, an integrated glucose meter is provided which, if necessary, can be used by several users, a specific set of data being provided for each user. Consequently, it is ensured that each user can be treated in an optimal way on the basis of individual data.

The software can execute control functions relating to the operating parameters. In this way, it is ensured that the user need not influence the operating parameters, thus ruling out the possibility of incorrect treatment.

The user identification can be software-controlled and also carried out mechanically. A software-controlled option permits input of a password or similar code which constitutes an effective protection against unwanted contamination of the glucose meter. The password can be used to secure the identity of the user, but it is not absolutely essential. A mechanical user identification can be effected by way of a characteristic pressure profile which is created when a user presses the glucose meter against his body. In this way, people who have no knowledge of software are also able to correctly use the glucose meter. It is conceivable that the user identification can be executed through contact between the user's body and any part of the glucose meter. In particular, it is conceivable that the body contact is effected by placing a finger on an appliance opening which is adapted to the conditions of said part of the body and which preferably can be designed as a cone or in another configuration with corresponding function. This embodiment is advantageous because in this way an element is activated which is essential for taking a sample of blood. Via an appliance opening of this kind or a support surface with several sensors, it is possible to establish a pressure profile on the basis of which a user identification by skin contact can take place.

The user identification can also be executed through activation of a keyboard. The provision of a keyboard permits exact entry of numerical codes or passwords. The user identification can also be realized, however, by activating a touch screen. A touch screen permits direct communication of the user with the glucose meter and additionally represents a graphic aid helping to instruct the user in the use of the glucose meter in an optimal manner.

The user identification can also be executed through activation of a mouse or of a joystick. These aids are readily available on the market and can be coupled particularly easily to the software. The user identification can also be executed through transfer of data on a smart card. The use of a smart card is advantageous since most health authorities provide such smart cards. It is thus conceivable for health authorities to acquire information on whether the user has used the glucose meter at regular intervals. It is also conceivable for doctors to have access to these data. This permits particularly effective treatment of a patient suffering from diabetes.

The sampling device can also be controlled by the software. This ensures that a sampling procedure takes place only when the user identification has been completed.

The sampling device can be assigned an exchangeable lancet which can be removed from a lancet magazine allocated to the sampling device. This ensures that infected lancets can be replaced or can be cleaned. This largely eliminates the risk of infection for different users of a glucose meter. Different lancets can be assigned to different users. This ensures that users with different skin characteristics can be treated with different types of lancets. The lancet is automatically replaced after a predetermined number of uses. This ensures that blunted lancets are removed after a certain cycle and that undefined injuries to a user are thus avoided.

The analysis device can for example be controlled by the software. This ensures that an analysis of the blood sample takes place only when the user is identified. Misuse of data is to this extent ruled out. Optical measurement instruments can also be assigned to the analysis device. The provision of optical measurement instruments is advantageous in that the flow of blood and the blood picture can be recorded visually.

The delivery device can be controlled by the software. The test strip or test support is transported by means of the delivery device. In this way, for example, it is possible to regulate the wetting time, with the test strip or test support being driven to the skin opened by the lancet, taking up blood and waiting for complete wetting of the test value, for example, before the test strip or test support is moved away again from the skin. The delivery device transports the test strip or test support sooner or later, depending on the wetting time, away from the skin opened by the lancet. The controllability of the delivery device by the software permits adaptation of the delivery parameters entirely as a function of the speed of flow of blood in the respective user.

The glucose meter can also be provided with at least one display permitting communication between the glucose meter and the user. In this way, an optimal exchange of information between glucose meter and user is made possible. It is conceivable for the glucose meter to inform the user that consumable materials such as test support, lancets or the like have been depleted. It is also conceivable for the glucose meter to request the user to enter user-specific data so that user identification is ensured before use of the glucose meter.

The database can store not just the measurement values together with date, time and various flags, but also diverse user data such as user identification, user name, access authorization and, optionally, a password. In addition, together with the specific user data, the optimal parameters for blood sampling can also be stored. Moreover, access can be made to an inventory listing all the consumable materials, for example lancets.

Use of the appliance could be preceded by the user entering information as to whether he is the sole user of the glucose meter or whether a multi-user mode is desired. The multi-user mode enforces the safety feature of user identification prior to blood glucose measurement.

The databases can also optionally be used to store measurement values or diary entries relating to bread unit tables, dietary habits, or amounts of insulin to be administered.

### BRIEF DESCRIPTION OF THE DRAWINGS

The above-mentioned aspects of the present invention and the manner of obtaining them will become more apparent and the invention itself will be better understood by reference to the following description of the embodiments of the invention taken in conjunction with the accompanying drawings, wherein:

FIG. 1 is a schematic view of a pressure-sensitive actuating member which interacts with several mechanical switches, according to one embodiment of the present invention;

FIG. 2 is a graph of a pressure profile;

FIG. 3 is a fragmentary schematic view of a finger placed on a finger cone of an appliance in accordance with an embodiment of the present invention;

FIG. 4 is a graph of a pressure profile compared to a stored pressure profile in accordance with an embodiment of the present invention;

FIG. 5 is a schematic view that illustrates an optical measurement device designed for a chemical color reaction in accordance with an embodiment of the present invention;

FIG. 6 is a schematic view of a measurement device comprising a capillary arrangement in accordance with an embodiment of the present invention;

FIG. 7 is a schematic view of a measurement device comprising a camera with downstream software in accordance with an embodiment of the present invention;

FIG. 8 is a flowchart illustrating user identification before a measurement is carried out in accordance with an embodiment of the present invention;

FIG. 9 is a flowchart illustrating the initializing of a multi-user mode appliance in accordance with an embodiment of the present invention;

FIG. 10 is a flowchart illustrating a use cycle of an appliance in accordance with an embodiment of the present invention; and

FIG. 11 is a flowchart illustrating a learning mode of an appliance in accordance with an embodiment of the present invention.

Corresponding reference characters indicate corresponding parts throughout the several views.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

An analysis appliance according to an exemplary embodiment is described in more detail below using the example of a portable glucose meter for glucose determination. This analysis appliance is generally used by untrained users and is designed so that it can be pressed against various parts of the body for taking a blood sample.

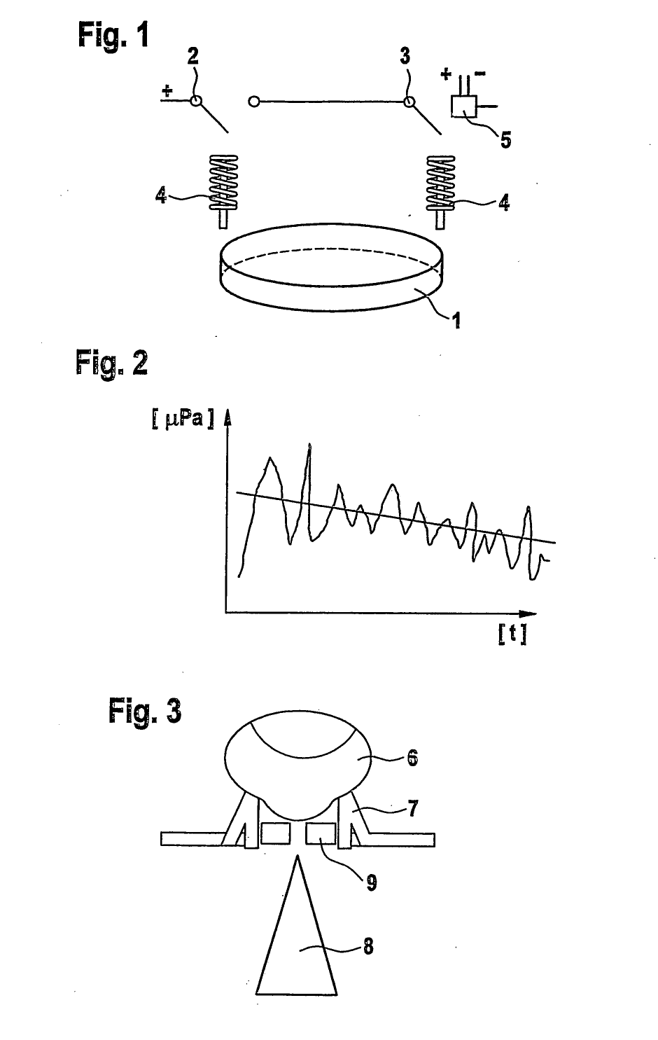

Referring to FIG. 1, an actuating member 1 is operatively connected to mechanical switches 2, 3. When pressure is applied to the actuating member 1, the switches 2, 3 close a contact. The actuating member 1 rests on an elastic abutment in the form of a spring 4. A pressure sensor 5 is assigned to the actuating member 1 and to the switches 2, 3. The number of switches can vary from 1 to n, depending on the design.

FIG. 2 illustrates applied pressure versus time. This diagram is recorded by the pressure sensor 5. The software records whether all the resistances afforded by the springs 4 are overcome by the pressure applied by the user at the measurement point. The recorded profile represents the basic data for the current user, and these basic data are stored in a database which can be accessed at the time of later measurements. The pressure profile can be the image of the personal handling of the appliance by the user or can be an impression profile pertaining to a part of the body, for example finger, ball of thumb, arm or forearm. The pressure profile is dependent on the handling of the analysis appliance by the respective individual user, depending on whether the user is right-handed or left-handed, whether he is familiar with the use of the appliance or unfamiliar with it. The skin curvature also has an influence on the pressure profile, depending on whether, for example, the appliance is placed against a stocky arm or a somewhat thin arm.

FIG. 3 illustrates a finger 6 which is pressed onto a finger cone 7. A lancet 8 provides the finger 6 with a wound in order to collect a blood sample. The lancet 8 can be positioned via a spacer 9 in such a way that the blood sampling point is disturbed, that is to say pressed, as little as possible, and the zero point for the penetration of the lancet 8 is obtained as exactly as possible. The spacer 9 also represents a user-related parameter.

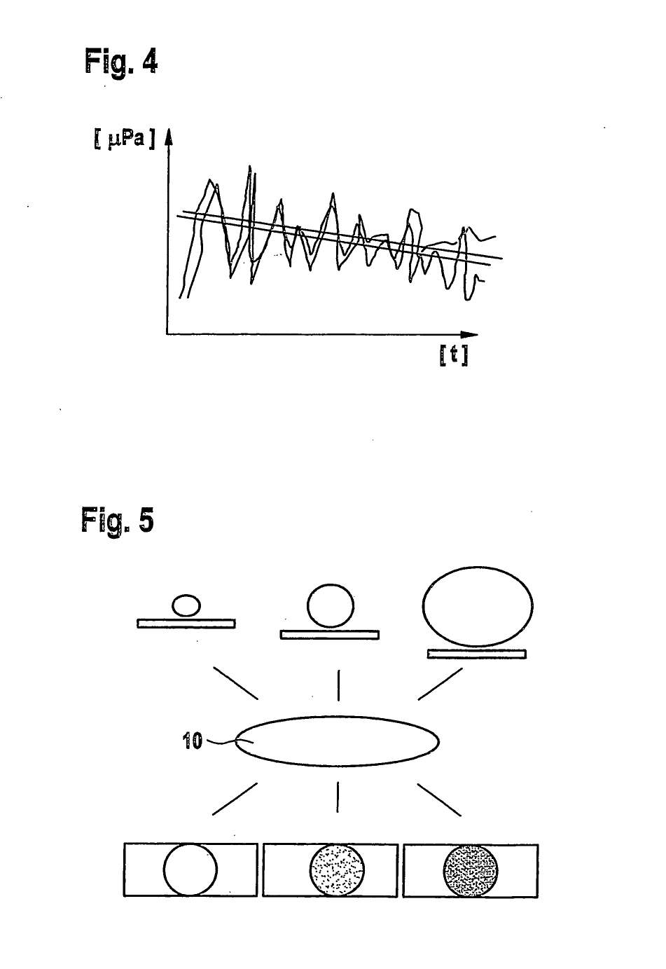

FIG. 4 illustrates a diagram in which the applied pressure is plotted against time. In this diagram, a pressure profile is shown compared with a previously stored pressure profile. With several pressure sensors, it is also possible, for example, for a curvature of a certain part of the body and/or the user to be registered. This can be done, for example, by means of a curvature profile of a fixed part of the body, for example the arm, an individual contact pressure and an optical position detection, so that the pressure sensor can also permit identification of the respective user. If the pressure profiles agree, the trigger mechanism for the lancet 8 is released. When all the conditions demanded by the appliance software are satisfied, the lancet 8 is released to pierce the skin. The lancet 8 is then triggered either automatically or manually, depending on the setting chosen by the user. After the lancet 8 has been triggered, it either moves to a park position or is placed in a corresponding magazine. The lancet 8 can be cleaned in the park position.

Referring to FIG. 5, illustrated is an optical measurement device in which a chemical color reaction on a test field 10 is monitored. From the intensity of a coloring of the test field 10, or from detection of a color change, it is possible to compute the volume of blood applied.

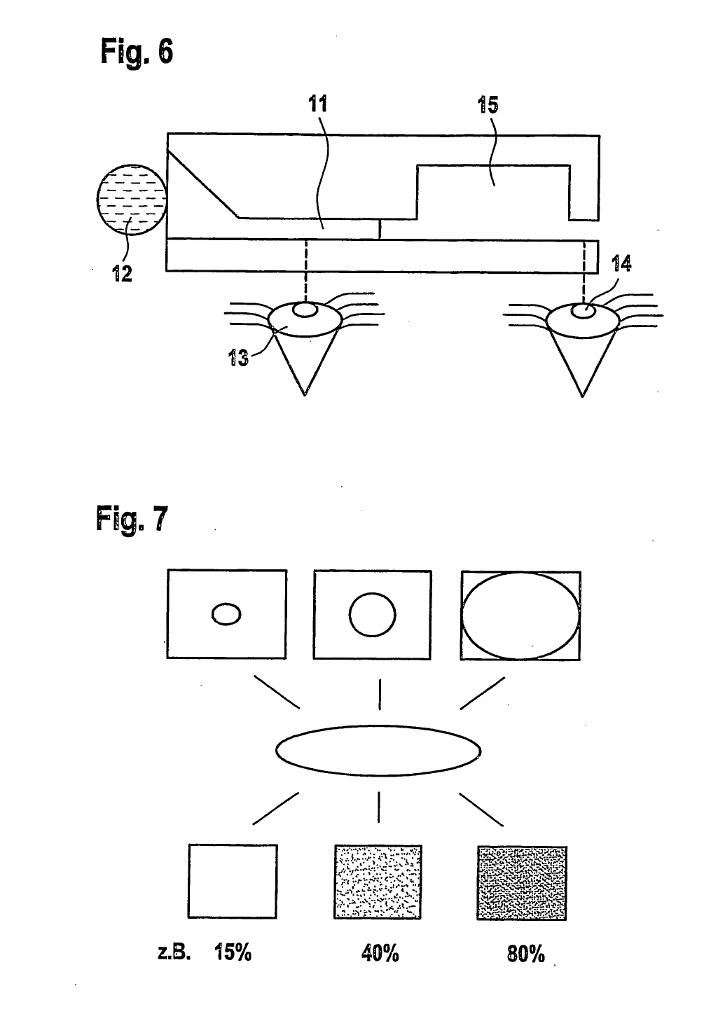

Turning now to FIG. 6, a measurement device is shown with a capillary 11 through which blood 12 flows. A sensor 13 checks the through-flow, and a sensor 14 monitors a test field 15. The sensor 13 detects whether blood has been taken up. The sensor 14 checks whether blood has flowed through the whole test area.

FIG. 7 shows a measurement device in which a camera records an image of the drop of blood after the skin has been punctured. Software continuously evaluates the image and determines the volume of blood expelled in the time t.

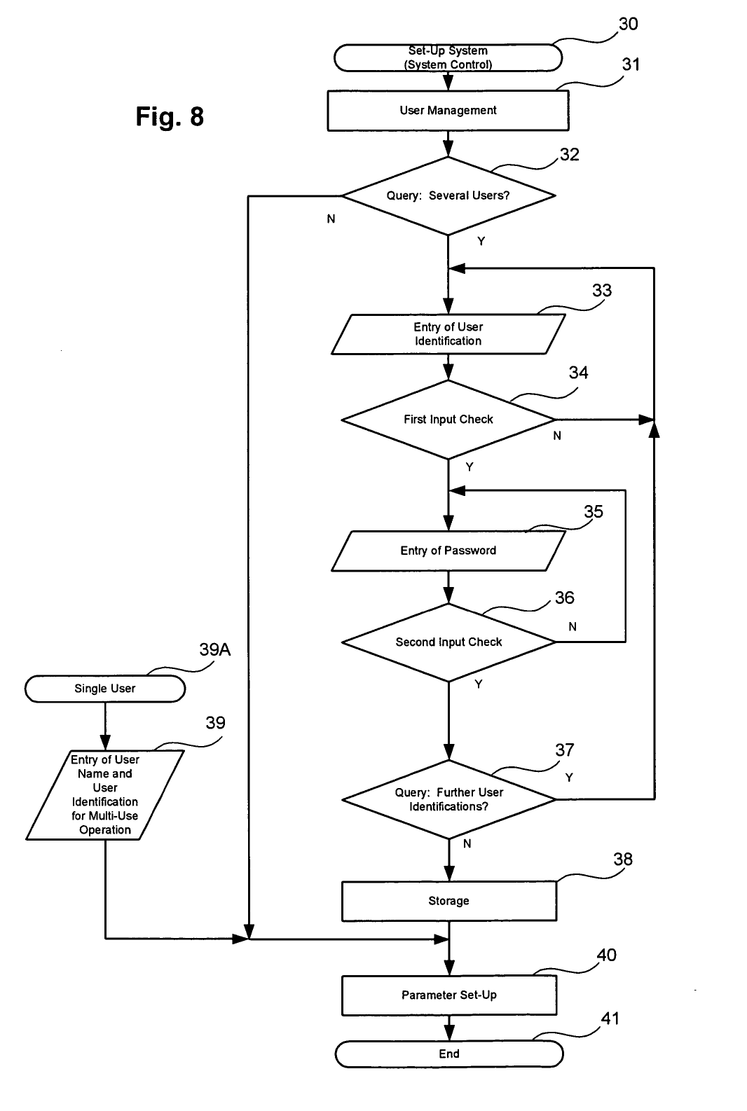

FIGS. 8-10 illustrate the implementation of a user identification procedure in a measurement operation. The measurement operation can be divided roughly into software operations and hardware operations. The software operations include database functions, control functions, and the management of user interfaces, e.g. keyboards, displays and the like, and the evaluation of the measurement results obtained. The hardware operations include the mechanics of blood sampling concerning management of magazines, lancets and drives, and the mechanics of the blood recovery, represented by the handling of magazine test strips and drive and the management of the measurement in respect of optics and LEDs.

The measurement operation outlined in the flowcharts in FIGS. 8-10 shows clearly that the proposed analysis appliance, e.g. a glucose meter, is designed for more than just a single untrained user. A database now not only stores the measured values, optionally supplemented by date, time and various flags, but also other user data, for example the user identification, represented by the user's name and an access authorization, represented by a password. These can be stored in the database, if appropriate together with other specific user data, such as the optimal parameters for blood sampling. Moreover, an inventory function can be provided for all the consumable materials, for example the lancets used in the glucose meter. The user interface of the analysis appliance now permits not only the output of measured values, optionally supplemented by date, time and various flags, but also dialogues with the glucose meter user. By way of the user interface, it is possible for the user to input user-specific data.

According to the flowchart shown in FIG. 8, the untrained user is first required to inform the appliance, in this case a glucose meter, of who the single user of the glucose meter is or whether a multi-user operation is desired. Multi-user operation of one and the same glucose meter is possible because a user identification is integrated as a safety feature into the measurement operation during the glucose measurement. The user identification integrated into the measurement operation is implemented in the system control 30 (set-up) of the appliance as a subsection. According to FIG. 8, the user identification has the structure set out therein.

Via the system control 30 (set-up), the user of the glucose meter branches to user management 31. There, a query 32 is first made to determine whether the glucose meter has one or more users. The query is indicated by reference number 32 and can either be answered in the negative or in the affirmative. If the response to the query 32 is in the affirmative, the person using the glucose meter is asked to enter the user identification at 33. The entry 33 of the user identification is followed by a first input check 34 which checks whether the entry made by the user can be processed further. If this is not the case, the user is returned to entry 33 of the user identification.

In the affirmative case, the user is asked to enter 35 his password. The entry 35 of the password is also followed by an input check, in the present case a second input check 36, in which the password entered by the user is checked. If the entry is not correct, a request is again made to enter the password 35. If it has been correctly entered, the operation continues with a query 37 for further user identification 37. If further user identifications are to be entered, the operation returns to entry 33 of the user identification; if the response is negative, the input user data are stored in a database, which takes place in step 38. This is followed by a parameter set-up 40 according to the user query where, in the case of one user, the user identification is deactivated and, in the case of several users, the user identification is activated. From the parameter set-up 40, the operation branches to the end 41 of the subsection which is integrated in the system control 30.

If the query 32 located downstream of the user management 31 and concerning use by several users is answered in the negative, the further sequence of questions is directly bypassed and the operation goes directly to parameter set-up 40.

As an optional possibility, provision is also made for entry 39 of a single user 39A name and user identification in the case where the proposed integrated glucose meter is not used in multi-user operation. In this way it is possible, for example, to assign a lost appliance to the user. In this case, the user identification can be optionally extended, for example in order to be able to enter an address or telephone number.

The user can enter freely chosen user names at the entry 33. For security reasons, provision can be made for the entry 35 of a password, but this is an optional possibility. The user identification can be entered via one or more keys, or via small keypads integrated in the appliance, or via an optionally connectable keyboard, either numerically or alphanumerically, or in combinations of these. It is also possible to enter data via a drag ball, a mouse or a joystick which are either integrated in the appliance or can be connected to it externally. Moreover, data can be entered by voice control, by touch screen, or by selection made by finger or pin. In addition, it is possible to perform user identification via a sensor, for example for identification of fingerprints, in which case the entry step 35 for a password could be omitted. It is also possible to use a user-specific smart card to feed the user identification into the glucose meter in a manner that can be read out.

After the query in respect of the user-specific identifications or user-specific password, the selected option is placed in the database, for example whether a multi-user operation is desired (multi-user=true, or multi-user=false). If they have been entered, the user identification or user identifications and the associated passwords are stored in the database. It is possible to dedicate a specific data area in the database for each user of the glucose meter, in order to place additional user-specific data, measured values or diary entries there, for example the bread units table, the administered quantities of insulin and dietary habits. In this way, it is also possible for the user-specific sampling parameters determined by the learning mode to be assigned unambiguously to the current user. To simplify handling during user identification, the password could also optionally be turned off.

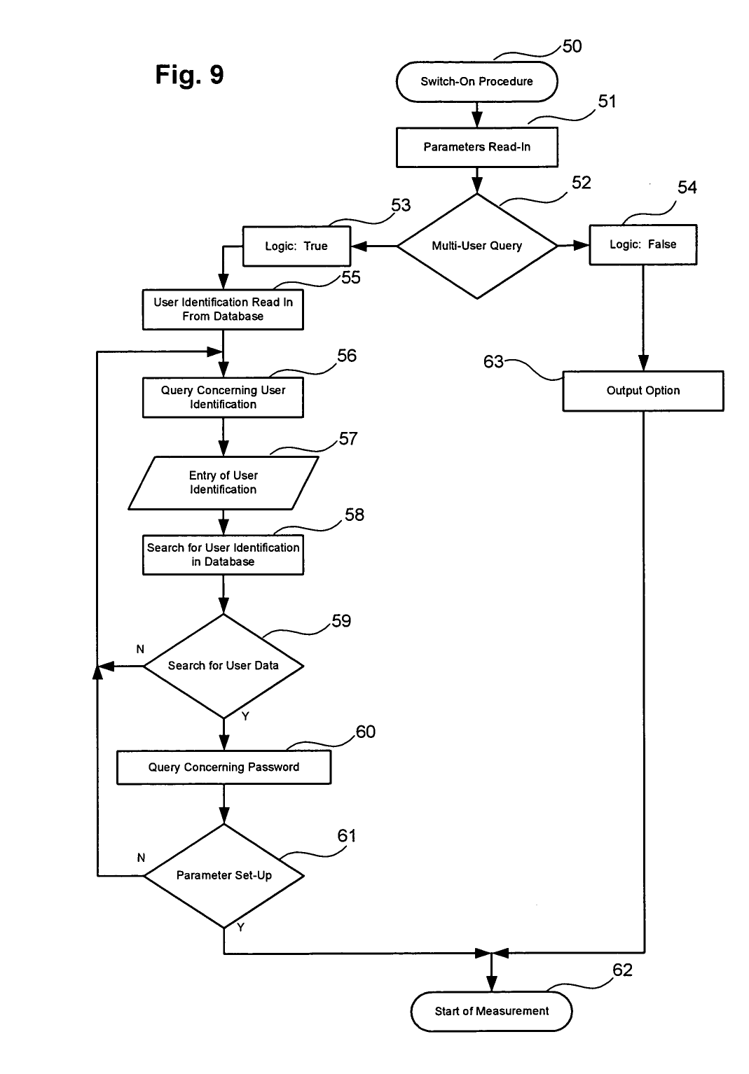

After it has been switched on, the analysis appliance is initialized and, during the switch-on procedure, all the appliance-specific and database-specific parameters are also read in. This also includes the variable which identifies the multi-user mode, i.e. multi-user=false or true. If, for example, multi-user=false is read in, optionally input user data can nevertheless be read in, for example to permit allocation of a lost appliance or of a second appliance within a user circle. If the variable is multi-user=true, all the stored user data with the associated databases are initialized and read in. Depending on the state of the multi-user variable, the subsequent operations in the user procedure are executed with the hardware control, if appropriate with adaptation. The structure of the initializing of the multi-user mode can be seen from the flowchart shown in FIG. 9.

After a switch-on procedure 50, parameters are read in 51. The parameters also include the variable “multi-user” to be read in. In a query 52 for the variable “multi-user,” the latter is assigned either the value True 53 or the value False 54.

In the case where the variable “multi-user” assumes the value False 54, the operation branches to an optionally implemented user identification read-in 63, from which the start 62 of the measurement can be directly initialized.

In the case where the variable “multi-user” assumes the value True 53, the user identification is read in 55 from the database. This is followed by a user identification query 56 which is input at 57. The user identification input at 57 is targeted in a search 58 in the database to an already existing set of data. If the search 59 was successful, the operation is continued with a query 60 concerning the password. In the case where the search 59 was unsuccessful, the operation is returned to the query 56 concerning user identification.

After the optional password query 60, the latter is examined for its authenticity in a check step 61. If the check 61 points to the authenticity of the password, the operation is branched to the start 62 of the measurement; otherwise the operation returns to a point before the query 56 concerning user identification.

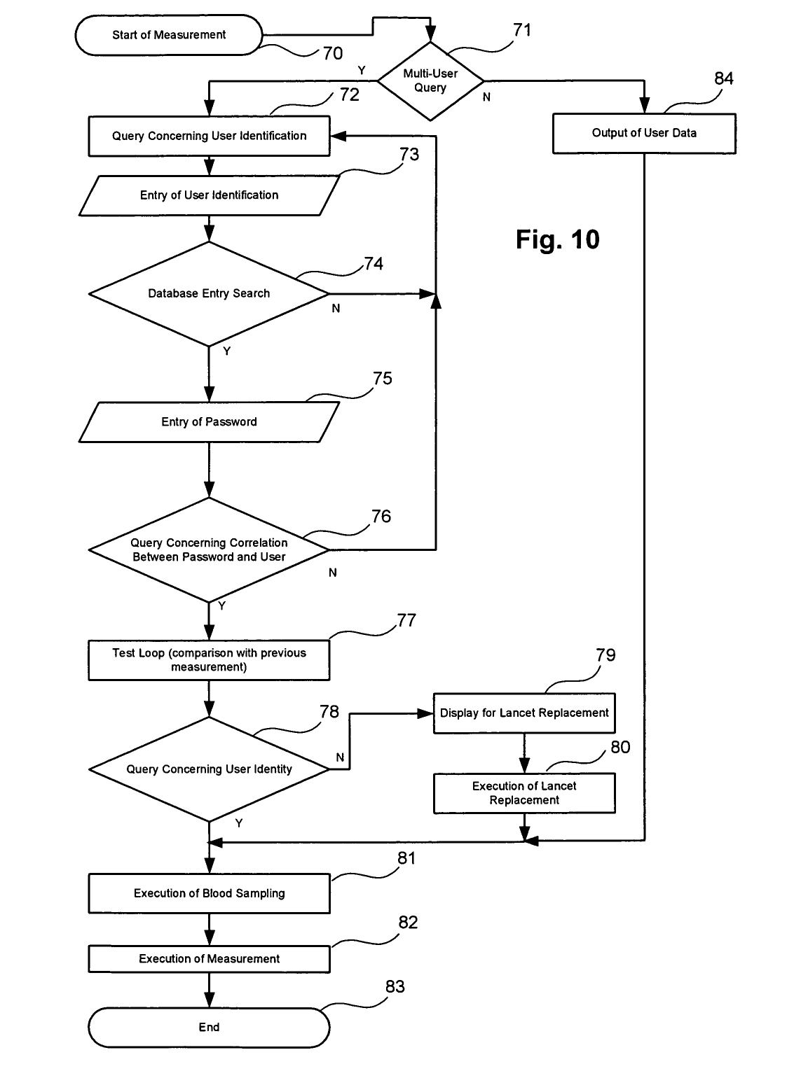

The function of a user identification as a security feature of a glucose meter, as outlined roughly in the flowchart according to FIG. 10, is used when the user starts a blood glucose measurement operation 62. If the “multi-user” variable assumes the value True 53, the actual measurement is preceded by the user identification concerning user name and an optional password. If the appliance software recognizes that the user has not changed since the last measurement, the lancet contained in the glucose appliance is not replaced. Instead, a check is made to establish whether the maximum desired number of uses with the lancet in question has been reached or whether this is not the case. The use cycle is fixed by the user in the appliance set-up and permits a corresponding multiple use of one and the same lancet, for example to cut down on material costs. An automatic lancet replacement can also take place after a number n of uses defined by the user. If the counter status counting the use of the lancet is still smaller than the defined number of use cycles, the lancet does not have to be replaced, and blood coagulation takes place, followed by blood glucose measurement 62. If the maximum number of use cycles for the lancet in question is reached, the latter is replaced even if the user remains the same. For reasons of clarity, the use cycle and accompanying functions such as testing of the inventory function are not included in the flowchart according to FIG. 10. It is clear from the flowchart according to FIG. 10 that, after the start 70 of the measurement operation, the operation branches to the multi-user query 71. The program proceeds as a function of whether the “multi-user” variable assumes the value True 53 or False 54.

In the case where the variable “multi-user” assumes the value false 54, the operation branches to the optional possibility of output 84 of the user data. From there, the program advances to execution 81 of the blood sampling, which is followed by execution 82 of the blood glucose measurement 82 before the program reaches the end 83.

If, by contrast, the “multi-user” variable assumes the value “True”, as indicated by reference number 53, the program continues to a query 72 concerning user identification. At 73, the user identification is entered, and this is checked in a search 74 for the presence in a database. If this is not the case, the operation returns to the query 72 concerning user identification; otherwise the optional password is entered 75. After the password is entered 75, a query 76 checks whether the input password matches the user or not. If not, the operation returns to the query 72 concerning user identification; if it is the case, a test loop 77 determines whether the current user of the glucose meter is the same user who performed the last measurement with this appliance. In the negative case, the query 78 concerning user identity checks whether a lancet replacement 79 is necessary. This is done, if necessary, in step 80.

If it emerges from query 78 concerning user identity that it is the same user who used the glucose meter in the last measurement, blood is collected in step 81, and this is followed, according to step 82, by execution of the measurement. At the end of the measurement, the operation is branched to the end 83.

By means of the procedure illustrated according to flowcharts 8-10, a glucose meter can be prepared which permits database access designed for a multi-user mode. The implemented user identification permits greatly enhanced hygiene protection, or protection against infections, when the glucose meter is used by several users. If, on the basis of the user identification entry or password entry, the glucose meter detects that different users are involved, a user change is detected, so that a lancet replacement can be necessarily performed between two successive measurements. In the context of the user identification shown in the flowcharts 8-10, further functions can be provided, for example, system cleaning, user-specific welcome messages or welcome images in glucose meters with displays. Moreover, the database access can be extended so that the last measured values can be automatically displayed. In addition, more extensive access to databases is provided by the fact that an electronic diary can be automatically retrieved and instructions can be given as to what quantity of insulin must be administered at what time of day.

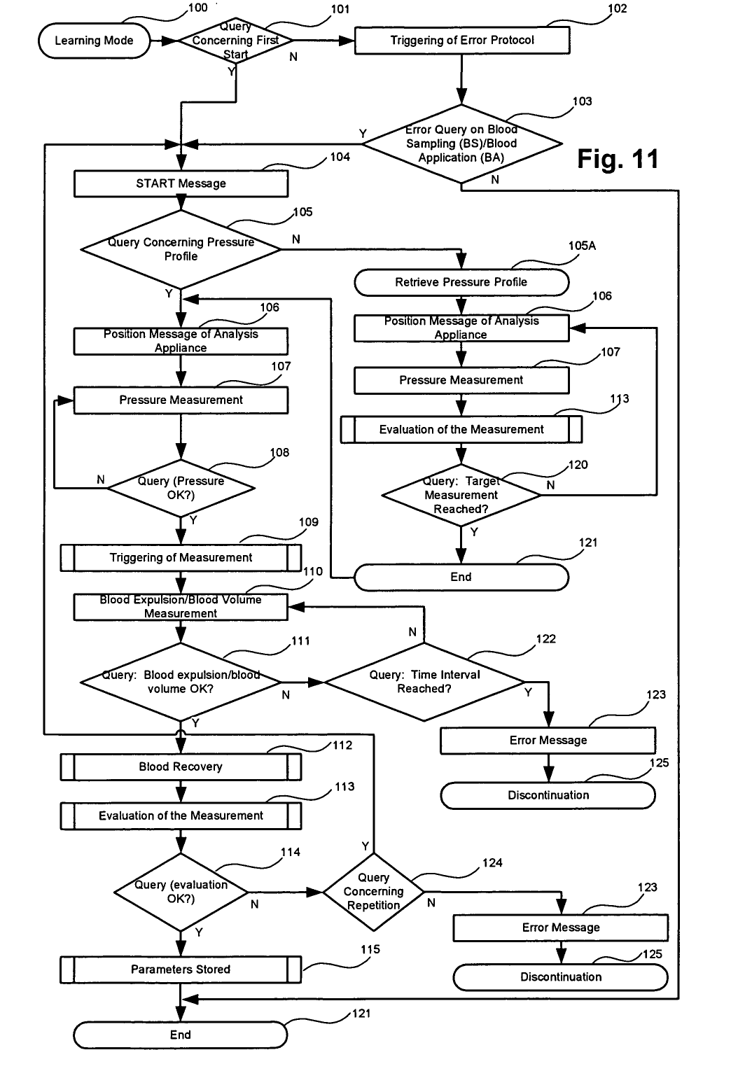

FIG. 11 shows a flowchart of a learning mode. A learning mode 100 for a glucose meter comprises a query 101 as to whether this is or is not a first start. If the response to query 101 is negative, then an error protocol 102 is read out. The read-out of an error protocol 102 is followed by an error query 103 in which a check is made on whether or not an error has occurred in the context of the blood sampling or in the context of application of blood to a test support. An error function can arise if, for example, the blood flow characteristics of a user already known to the system have changed. If the response to the query 103 is negative, the operation is branched to the end 121 of the flowchart. If, by contrast, the response to the query 103 is affirmative, the operation is branched to a start message 104.

If the query 101 as to whether this is a first start-up of the appliance is answered in the affirmative, the operation is branched directly to the start message 104. This is followed by a query 105 concerning the knowledge of a pressure profile. If the response to this query is affirmative, the operation is branched to a position message 106; if it is answered in the negative, a pressure profile is retrieved 105A and a position message 106 of the appliance takes place. Thereafter, a pressure measurement 107 is carried out, followed by an evaluation 113 of the measurement. The evaluation 113 of the measurement is followed by a query 120 on whether the target measurement is reached. If the response to query 120 is affirmative, the operation is branched to the end 121. By contrast, if the response to query 120 is negative, the operation returns to the position message 106 of the appliance. If, within the query 105 concerning the pressure profile 105, the latter is known, the position message 106 of the appliance takes place immediately, followed by a pressure measurement 107. The pressure measurement 107 is followed by a query 108 which checks whether it is the correct pressure. If this is not the case, the operation returns to the pressure measurement 107, and the pressure applied by the user of the glucose meter to the part of the body concerned is measured once more. If a correct pressure is determined within query 108, the measurement is triggered 109. In the context of the measurement 109, the blood expelled and blood volume is measured 110. This includes a query 111 as to whether the speed of expulsion of the blood or the volume of the blood is or is not correct. If negative, the operation branches from query 111 to a further query 122 which checks whether the measurement time interval is exceeded. If this is not the case, the operation returns to the blood expulsion and blood volume measurement 110. By contrast, if it is found, in query 122, that the measurement time interval is exceeded, the operation branches to an error message 123, which is followed by a discontinuation 125.

If, in the context of query 111, it is found that the volume of blood expelled is correct or that the amount of blood is sufficient for the evaluation, the operation branches to blood recovery 112, which is followed by an evaluation 113 of the measurement. In the context of the evaluation 113 of the measurement, a query 114 checks whether the evaluation has been implemented correctly. If this is not the case, the operation branches to a query 124 in which it is determined whether the measurement is to be repeated. If the response is affirmative, the operation returns to the start message 104; if negative, there is an error message 123, followed by a discontinuation 125.

By contrast, if it is found in the context of query 114 that the evaluation was error-free, the parameters obtained are stored in a parameter memory 115, which is followed by the end 121 of the program run.

In the learning mode 100 in which the analysis appliance proposed according to the invention can be operated, it is possible, depending on the number of users, for a large number of user-specific parameters determined to be stored in a database. Upon renewed use of the analysis appliance by a user who has already used the analysis appliance proposed according to the invention, the user-specific parameters that have already been stored can be used again. The user identification ensures that the analysis appliance completely or partially permits use by the particular person, so that it is possible to limit the circle of users to a selected circle of persons.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

### LIST OF REFERENCE NUMBERS

1 actuating member
2 switch
3 switch
4 spring
5 pressure sensor
6 finger
7 cone
8 lancet
9 spacer
10 test field
11 capillary arrangement
12 blood
13 sensor
14 sensor
15 test field
30 set-up (system control)
31 user management
32 query: several users?
33 entry of user identification
34 first input check
35 entry of password
36 second input check
37 query: further user identifications?
38 storage
39 entry of user name and user identification for multi-user operation
39A single user
40 parameter set-up
41 END
50 switch-on procedure
51 parameters read-in
52 multi-user query
53 logic: True
54 logic: False
55 user identification read in from database
56 query concerning user identification
57 entry of user identification
58 search for user identification in database
59 search for user data
60 query concerning password
61 parameter set-up
62 start of measurement
63 output option
70 start of measurement
71 multi-user query
72 query concerning user identification
73 entry of user identification
74 database entry search
75 entry of password
76 query concerning correlation between password and user
77 test loop (comparison with previous measurement)
78 query concerning user identity
79 display for lancet replacement
80 execution of lancet replacement
81 execution of blood sampling
82 execution of measurement
83 end
84 output of user data
100 learning mode
101 query concerning first start
102 triggering of error protocol
103 error query on blood sampling (BS)/blood application (BA)
104 START message
105 query concerning pressure profile
105A retrieve pressure profile
106 position message of analysis appliance
107 pressure measurement
108 query (pressure ok?)
109 triggering of measurement
110 blood expulsion/blood volume measurement
111 query: blood expulsion/blood volume ok?
112 blood recovery
113 evaluation of the measurement
114 query (evaluation ok?)
115 parameters stored
120 query: target measurement reached?
121 end
122 query: time interval reached?
123 error message
124 query concerning repetition
125 discontinuation

What is claimed is:

1. An analysis appliance for analysis of blood samples, comprising:
a sampling device for taking blood samples;
an analysis device;
a delivery device for transferring the blood samples from the sampling device to the analysis device; and
at least one pressure sensor for sensing and recording a pressure profile of a particular user, the analysis appliance being configured to compare the recorded pressure profile with a predetermined pressure profile stored in a database for the particular user.

2. The analysis appliance of claim 1, wherein the analysis appliance is configured to identify a particular user from the comparing of the recorded pressure profile to the predetermined pressure profile and at least partially permit use of the analysis appliance by the particular user, the analysis appliance being further configured to adapt operating parameters concerning sampling, transfer of the sample to the analysis device or analysis of the sample to the particular user.

3. The analysis appliance of claim 1, wherein the appliance is configured to adjust one or more of the operating parameters by automatic execution of one or more test measurements, the test measurements being determinable by a software control and stored in a database and retrieved.

4. The analysis appliance of claim 1, wherein the pressure sensor comprises a trigger.

5. The analysis appliance of claim 1, wherein the pressure sensor comprises a pressure-sensitive actuating member.

6. The analysis appliance of claim 5, wherein the actuating member comprises an appliance opening or cone configured for automatic blood collection.

7. The analysis appliance of claim 5, wherein the actuating member is connected to one or more mechanical switches which close one or more contacts when pressure is applied to the actuating member.

8. The analysis device of claim 5, wherein the actuating member rests on a yielding or spring-cushioned abutment.

9. The analysis appliance of claim 5, wherein the actuating member is operatively connected to the sampling device, whereby the sampling device can be activated by applying pressure to the actuating member.

10. The analysis appliance of claim 1, wherein the sampling device comprises a lancet for puncturing skin.

11. The analysis appliance of claim 1, wherein the sampling device comprises a capillary.

12. The analysis appliance of claim 10, wherein the depth of insertion of the lancet is regulated by data recorded by the sampling device.

13. The analysis appliance of claim 1, wherein the operating parameters of the analysis appliance automatically adapt to a particular user prior to a measurement operation.

14. The analysis appliance of claim 13, wherein the adaptation of the operating parameters is executed by software which communicates with hardware and which is controlled via user interfaces.

15. The analysis appliance of claim 14, wherein the software accesses databases in which the operating parameters are stored.

16. The analysis appliance of claim 14, wherein the software executes control functions concerning the operating parameters.

17. The analysis appliance of claim 14, wherein the software is activated by a user identification.

18. The analysis appliance of claim 17, wherein the user identification is executed through body contact with the analysis appliance.

19. The analysis appliance of claim 17, wherein the user identification is executed by
activation of a keyboard, a touch screen or a mouse, or by transferring data from a smart card.

20. The analysis appliance of claim 17, wherein the sampling device is controlled by the software.

21. The analysis appliance of claim 1, wherein the sampling device comprises at least one exchangeable lancet.

22. The analysis appliance of claim 21, wherein the lancet is replaceable.

23. The analysis appliance of claim 1, wherein the sampling device comprises a removable lancet magazine which contains several lancets.

24. The analysis appliance of claim 1, further comprising software that controls the analysis device.

25. The analysis appliance of claim 24, wherein the analysis device comprises optical or electrochemical measurement instruments.

26. The analysis appliance of claim 1, wherein the delivery device is controllable by software.

27. The analysis appliance of claim 1, wherein the analysis appliance determines glucose concentration.

28. The analysis appliance of claim 27, wherein the analysis appliance comprises a portable glucose meter.

29. A method of analyzing blood samples using an appliance comprising a sampling device for taking blood samples, an analysis device, and a delivery device for transferring the blood samples from the sampling device to the analysis device, the method comprising:
(a) operating the appliance in a learning mode in which user-specific parameters are obtained by exerting pressure on the appliance by the user;
(b) storing the user-specific parameters in a database; and
(c) operating the appliance in an operational mode using the user-specific parameters.

30. The method of claim 29, further comprising switching on the appliance and reading a user identification from the database.

31. The method of claim 30, wherein the user identification comprises a query concerning a user-specific pressure profile.

32. The method of claim 30, further comprising entering a password and making a query concerning the correlation between password and user.

33. The method of claim 29, wherein step (a) comprises the user contacting a body part with a pressure sensor disposed on the appliance.

34. An analysis appliance for analysis of blood samples, comprising:
a sampling device for taking blood samples;
an analysis device;
a delivery device for transferring the blood samples from the sampling device to the analysis device;
an actuating member comprising a pressure sensor; and
software configured to correlate a signal from the pressure sensor to the identity of the user operating the analysis appliance or to the identity of the body part contacting the pressure sensor.

35. The analysis appliance of claim 34, wherein the actuating member comprises a switch.

36. The analysis appliance of claim 34, wherein the actuating member comprises an opening configured to receive a blood sample.

37. The analysis appliance of claim 36, wherein the actuating member comprises a spring.

38. The analysis appliance of claim 34, wherein the software is configured to correlate the signal from the pressure sensor to the identity of the user.
